(19) United States
(12) Patent Application Publication
(10) Pub. No.: US 2006/0089562 A1
(43) Pub. Date: Apr. 27, 2006

(54) Modified hickman-type catheter with embedded thermistor

(76) Inventor: D. Allen Gould, Omaha, NE (US)

Correspondence Address: LAW OFFICES OF ADAM H. JACOBS;PATENT ATTORNEY, SUITE 726, 1904 FARNAM STREET, OMAHA, NE, 68102, US

(21) Appl. No.: 11/256,233

(22) Filed: Oct. 21, 2005

Related U.S. Application Data
(60) Provisional application No. 60/621,097, filed on Oct. 22, 2004.

(51) Int. Cl. A61B 5/00

(52) U.S. Cl. 600/549

(57) ABSTRACT

A long-term internal temperature measurement device includes a silastic, tunneled right-atrial catheter having a distal end adapted to be directed into the superior vena cava of a patient to reside within one of the superior vena cava and the right atrium of the patient's heart, an outer end extending outside of the patient and a cuff positionable beneath the skin of the patient for generally preventing bacterial growth up the catheter. A thermistor device is mounted within the catheter, specifically within the distal end of the catheter, the thermistor device operative to measure the temperature of blood flowing through the patient's right atrium or superior vena cava. Finally, an electronic display device is in information transmission connection with the thermistor device and further is operative to display the internal temperature of the patient as measured by the thermistor device within the patient's right atrium or superior vena cava.

### CROSS-REFERENCE TO RELATED PROVISIONAL PATENT

This application claims priority based on a provisional patent, specifically on the Provisional Patent Application Ser. No. 60/621,097 filed Oct. 22, 2004.

### BACKGROUND OF THE INVENTION

1. Technical Field

The present invention is directed to long-term internal temperature measurement devices for patients and, more particularly, to a silastic, tunneled right-atrial catheter (e.g., Hickman-type Catheter) having an embedded thermistor device operative to measure the temperature of blood flowing through a patient's right atrium or superior vena cava and an electronic display device in information transmission connection with the thermistor which is operative to display the internal temperature of the patient.

2. Description of the Prior Art

A critical element of patient monitoring is the need for accurate and timely measurements of patient temperatures. There are many different methods which are used to obtain patient temperatures, including the taking of temperatures via the mouth, ear, or rectum, each of which have inherent disadvantages. For example, oral temperature measurement is often inaccurate due to several factors, including inaccurate placement of the thermometer probe in the patient's mouth or from mouth breathing. Ear temperature measurements may be inaccurate due to ear infections and/or operator error such as failing to seal the thermometer tightly within the patient's ear canal which allows loss of the infrared heat from the tympanic membrane, which is what the thermometer is attempting to measure. Finally, and perhaps most importantly, rectal temperatures are usually contraindicated in oncology and other immunosuppressed patients since even a small tear in the rectal tissue resulting from the thermometer probe insertion can result in a life-threatening infection. There is therefore a need for an accurate and safe temperature measurement device which can be used to measure the patient's temperature without the deficiencies found in the prior art.

At least two temperature measurement devices designed for insertion into the body are currently being used in the medical field, specifically, the thermistor-equipped Foley catheter designed for measuring body temperature within the urinary bladder and the Swan-Ganz catheter, which is a flow-directed balloon-tipped, thermodilution pulmonary artery catheter, which is designed to measure the temperature of the patient's blood in the patient's pulmonary artery. Each of these devices provides for accurate measurement of the patient's body temperature, however, each of these devices include inherent deficiencies which restrict their effective use. For example, use of the Foley catheter is impractical for patients who do not otherwise need a Foley catheter, and as Foley catheters are frequently a source of urinary tract infections, the use of the Foley catheter is an unacceptable risk of infection in immunosuppressed patients. Regarding the use of the Swan-Ganz catheter, its primary purpose is to measure pressures within the heart and to measure blood flow through the heart, and the placement of the Swan-Ganz catheter within the body is extremely expensive and can result in pneumothorax, bleeding, infection, and other complications. Furthermore, because the insertion site for the Swan-Ganz catheter is a portal for infection, and further because the risk of infection increases the longer the Swan-Ganz catheter is left in place, it is standard medical procedure that the Swan-Ganz catheter be left in place for a maximum of three to five days. As thus described, there is currently no device which can provide a continuous, long-term temperature monitoring system for patients who also require long-term venous access.

Therefore, an object of the present invention is to provide an improved long-term internal temperature measurement device.

Another object of the present invention is to provide an improved long-term internal temperature measurement device which includes a silastic, tunneled, right-atrial catheter having a thermistor device mounted within the distal end of the catheter to measure the temperature of blood flowing through one of the patient's right atrium and superior vena cava.

Another object of the present invention is to provide an improved long-term internal temperature measurement device which further includes an electronic display device in information transmission connection with the thermistor device which is operative to display the internal temperature of the patient as measured by the thermistor device within one of the patient's right atrium and superior vena cava.

Another object of the present invention is to provide an improved long-term internal temperature measurement device which is generally non-invasive and which can quickly and easily be used to measure the internal temperature of a patient without requiring the insertion of a thermometer device into the patient's body.

Another object of the present invention is to provide an improved long-term internal temperature measurement device which may be left in a patient for upwards of one to three months as is often required with oncology and cardiology patients.

Finally, an object of the present invention is to provide an improved long-term internal temperature measurement device which is relatively simple to manufacture and is safe, effective and efficient in use.

### SUMMARY OF THE INVENTION

The present invention provides a long-term internal temperature measurement device including a silastic, tunneled right-atrial catheter having a distal end adapted to be directed into the superior vena cava of a patient to reside within one of the superior vena cava and the right atrium of the patient's heart, an outer end extending outside of the patient and a cuff positionable beneath the skin of the patient for generally preventing bacterial growth up the catheter. A thermistor device is mounted within the catheter, specifically within the distal end of the catheter, the thermistor device operative to measure the temperature of blood flowing through one of the patient's right atrium and superior vena cava. Finally, an electronic display device is in information transmission connection with the thermistor device and further is operative to display the internal temperature of the patient as measured by the thermistor device within one of the patient's right atrium and superior vena cava.

As thus described, the long-term internal temperature measurement device of the present invention provides numerous advantages over those devices found in the prior art. For example, because the thermistor-equipped catheter is designed for long-term retention within the patient's body, it becomes a relatively simple process to determine the internal temperature of the person outfitted with the present invention as opposed to the difficulties encountered in using other devices found in the prior art. Furthermore, because the thermistor device is integral with the Hickman-type catheter, insertion and removal of the catheter in the patient's body follows the same procedure as would be performed with a standard Hickman-type catheter and thus a user of the present invention need not learn an entirely new insertion method for the thermistor-equipped catheter of the present invention. Also, unlike other catheters in the prior art, the catheter of the present invention is designed to be left within the patient's body for an extended period of time, whereas other thermistor-equipped devices should not be left within the patient's body for an extended period of time, and thus temperature variations over an extended period can be measured to provide a better understanding of the functioning of the patient's body. Finally, because the thermistor device of the present invention is connected to an electronic temperature display device, reading of the internal temperature of the patient is rendered far easier than that encountered in using devices found in the prior art. It is thus seen that the present invention provides a substantial improvement over those devices found in the prior art.

### BRIEF DESCRIPTION OF THE DRAWINGS

FIG. 1 is a perspective view of the modified catheter and temperature readout of the present invention;

FIG. 2 is a detailed perspective view of the modified catheter of the present invention;

FIG. 3 is a detailed perspective view of the heart of a patient showing the positioning of the end of the modified catheter of the present invention within the superior vena cava or right atrium of the patient; and

FIG. 4 is a detailed perspective view of the temperature module of the present invention.

### DESCRIPTION OF THE PREFERRED EMBODIMENT

The present invention provides a modified Hickman-type catheter 10 which is intended for long-term placement in the patient's body. In the ordinary use of Hickman-type catheters, the distal end of the catheter would be tunneled under the patient's skin from an entrance site which is typically in the region of the sternum to the point of insertion into the vascular system which is usually under the clavicle into the subclavian vein. The distal end is then directed into the superior vena cava 54 until the distal end of the Hickman-type catheter resides within either the superior vena cava 54 or the right atrium 52 of the patient's heart 50. Because of the method of placement of the Hickman-type catheter into the body, the risk of infection is greatly reduced and further the Hickman-type catheter may be left within the patient for a long period of time as would be necessary for use in connection with oncology patients and other patients having a variety of chronic conditions whose management requires long-term venous access.

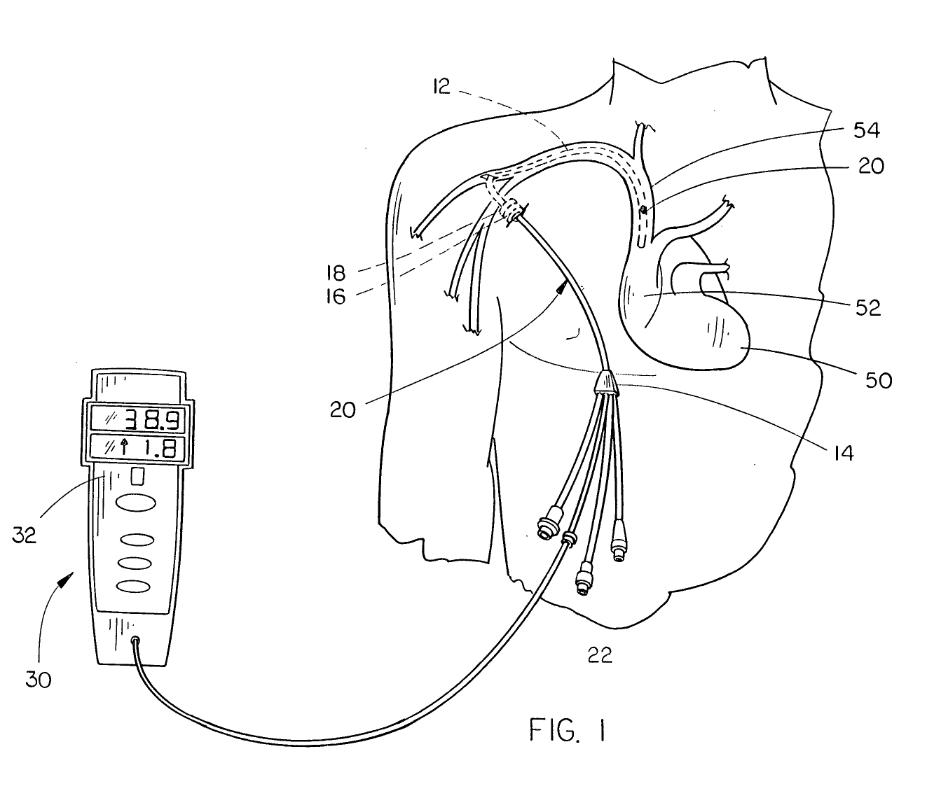

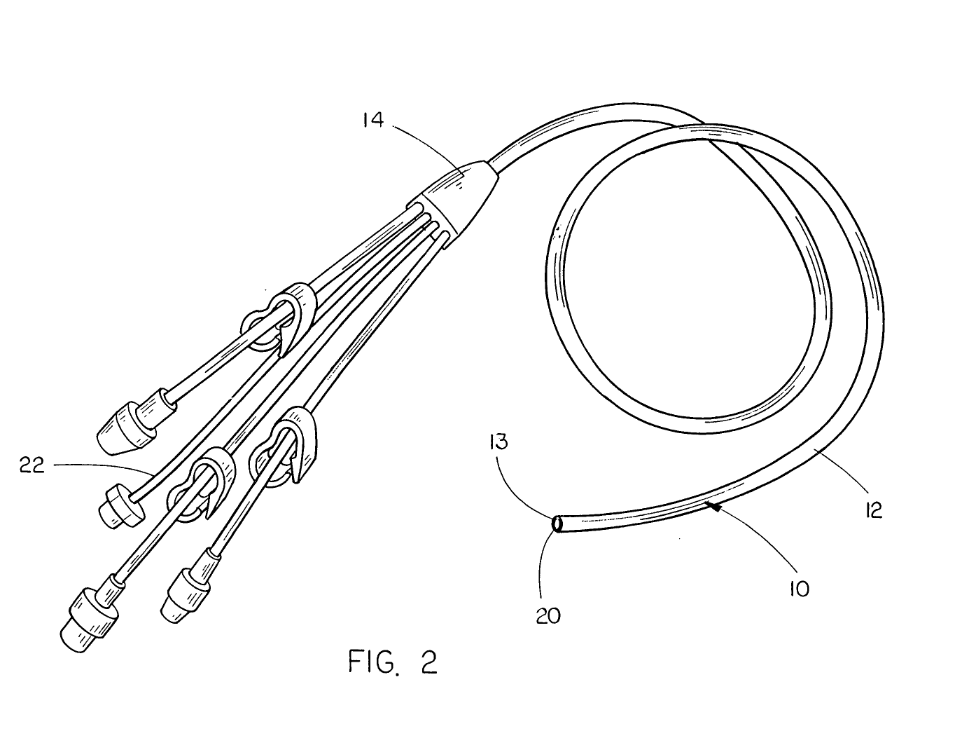

The modified Hickman-type catheter 10 of the present invention is shown best in FIGS. 1-3 as including an internal section 12 and an external section 14 which divides the internal elements of the catheter 10 into separately accessible units which permit connection of those separate elements to various operational devices of the kind commonly found and used in connection with Hickman-type catheters. In the preferred embodiment, the internal and external sections 12 and 14 would be constructed of a medical-grade radiopaque silicone to promote biocompatibility, and the catheter 10 may further include a tissue ingrowth cuff 16 and antimicrobial cuff 18 positioned on the internal section 12 to promote skin growth and grafting to the catheter while preventing the passage of infectious organisms through the insertion point.

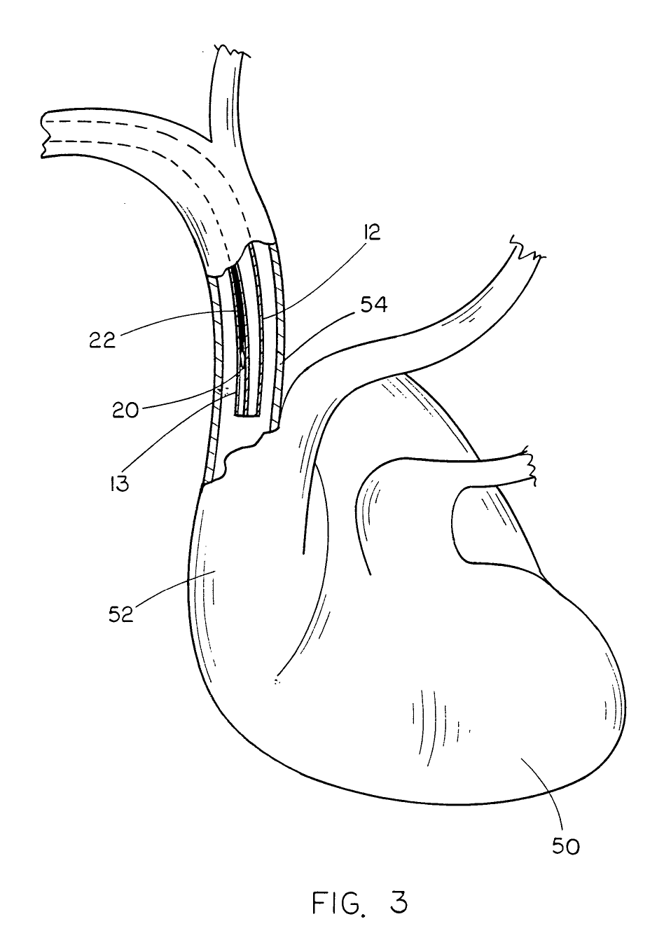

To this point, the present invention as thus described is similar to a standard Hickman-type catheter, however, the inventive elements of the present invention will now be described. Specifically, a thermistor unit 20 would be mounted within the outer wall 13 of internal section 12 generally adjacent the end of internal section 12, as shown best in FIGS. 1 and 3, such that the thermistor unit 20, when the modified Hickman-type catheter 10 of the present invention is placed within the patient's body, would be positioned within the superior vena cava 54 to permit accurate temperature measurement of the patient's blood. In the human body, the superior vena cava 54 returns blood from the patient's head and upper one-third of the body, and thus it is believed that the temperature of this blood is an accurate reflection of the temperature of the individual's body and thus would provide an accurate measurement of the patient's temperature.

Regarding the thermistor unit 20 itself, a thermistor is a resistor which measures temperature changes, relying on the change in its resistance to determine the specific temperature of the surrounding fluid. In the present invention, the thermistor unit 20 will provide an extremely accurate reading of the patient's temperature as the fluctuations in body temperature are well within the temperature measurement range of the thermistor unit 20, and it should be further noted that many different types of thermistor units 20 may be used with the present invention so long as the thermistor unit 20 provides an accurate and easily accessible temperature measurement. One or more electrical wires 22 would extend from the thermistor unit 20 through the internal section 12 and external section 14 of the modified Hickman-type catheter 10 of the present invention and be available for access and connection to a readout device 30 which will permit the temperature detected by the thermistor unit 20 to be read externally.

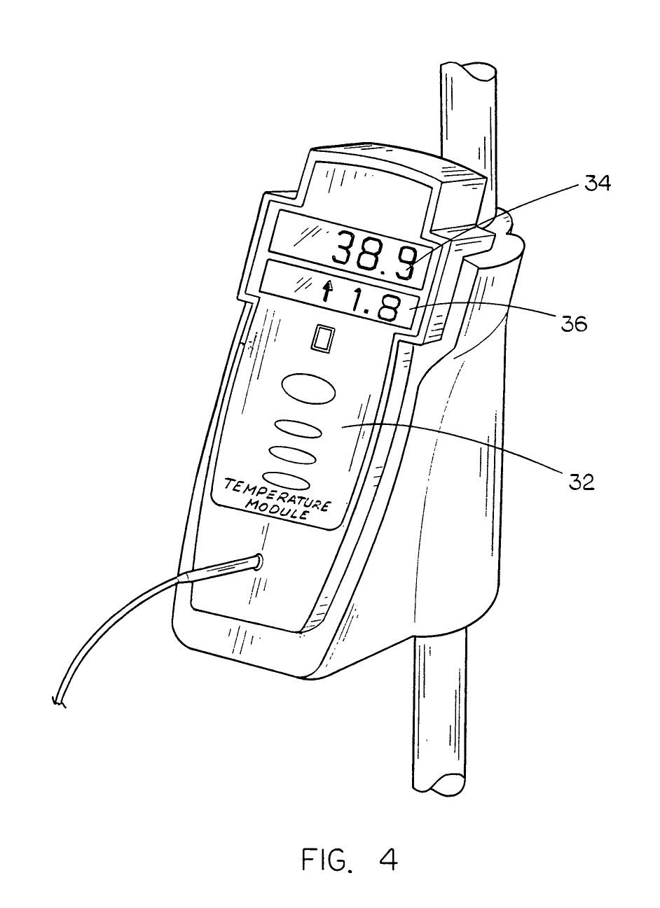

In the preferred embodiment, as shown in FIGS. 1 and 4, this display unit 30 would take the form of a temperature or thermometry module 32 which would provide a digital readout 34 of the patient's temperature and a graphical display 36 of the patient's temperature over time. In the preferred embodiment, the thermometry module 32 would also be programmed to permit the care giver to set upper and lower limits for the patient's temperature and, should those limits be exceeded, the thermometry module 32 would trigger an alarm to inform the care giver of the significant change in the patient's temperature. Additionally, the thermometry module 32 may include additional programming features which will compute the derivative of the temperature versus time curve, thereby measuring the rate of rise of the patient's temperature. This type of readout would alert care givers of a patient's rapidly rising temperature even before the temperature reached the set upper alarm limit which thus can provide notification to care givers of problems such as a transfusion reaction, reaction to a medication, or a developing infection. It is known that the best time for obtaining blood cultures, which are used to identify infectious organisms and to prescribe appropriate antibiotics for bloodstream infections, is when the patient's temperature is rising rapidly instead of when the patient has already spiked a fever. Also, the ability of the care giver to determine a rapid increase in patient temperature will permit the care giver to immediately take action to determine the cause of the rise in temperature and prescribe appropriate treatment, instead of having to wait until the temperature of the patient has already reached a dangerous level.

It should be noted that the development and use of a thermometry module 32, although preferred, is not strictly necessary to permit proper functioning of the present invention. For example, the thermistor unit 20 may be connected via the embedded wires 22 to a hand-held electronic thermometer, such as an IVAC model 2080A or model 2085, for intermittent temperature measurement. This alternative embodiment of the present invention would be a less expensive and more portable version which would permit measurement of a patient's internal temperature, albeit without many other functional features of the thermometry module 32, yet still would be superior to external temperature measurements such as measurements via the mouth, ear, or rectum. It is still preferred, however, that the modified Hickman-type catheter 10 of the present invention be connected to a specifically designed thermometry module 32 to take full advantage of the measurement features of the present invention which will greatly aid long-term patients requiring central venous access.

It is to be understood that numerous additions, modifications, and substitutions may be made to the modified Hickman-type catheter 10 of the present invention which fall within the intended broad scope of the above description. For example, the size, shape and construction materials for the elements of the present invention may be modified or changed so long as the functional features of the invention are neither degraded nor destroyed. Also, the positioning of the thermistor unit 20 within the outer wall 13 of internal section 12 may be modified or changed depending on the preferred positioning for the best temperature readings of the blood passing through the superior vena cava 54 of the patient. Furthermore, the exact nature of the connection between the thermistor unit 20 and display unit 30, described above as embedded wires 22, may be modified or changed should such modification prove beneficial to the patient using the modified Hickman-type catheter 10 of the present invention. Finally, the specific design features of the thermometry module 32 may be modified or changed depending upon the intended use of the present invention in connection with temperature measurement, and such modifications would be understood by those skilled in the art.

There has therefore been shown and described a modified Hickman-type catheter with embedded thermistor 10 which accomplishes at least all of its intended objectives.

I claim:

1. A long-term internal temperature measurement device comprising:
a silastic, tunneled right-atrial catheter including a distal end adapted to be directed into the superior vena cava of a patient to reside within one of the superior vena cava and the right atrium of the patient's heart, an outer end extending outside of the patient and a cuff positionable beneath the skin of the patient for generally preventing bacterial growth up the catheter;
a thermistor device mounted within said catheter, specifically within said distal end of said catheter, said thermistor device operative to measure the temperature of blood flowing through one of the patient's right atrium and superior vena cava; and
an electronic display device in information transmission connection with said thermistor device operative to display the internal temperature of the patient as measured by said thermistor device within one of the patient's right atrium and superior vena cava.

2. The long-term internal temperature measurement device of claim 1 wherein said thermistor device comprises a resistor operative to measure temperature changes within the body of the patient by measuring the change in resistance as the temperature varies to determine the specific temperature of the surrounding body fluid.

3. The long-term internal temperature measurement device of claim 1 further comprising electrically conductive wiring extending between and connecting said thermistor device and said electronic display device for transfer of temperature information from said thermistor device to said electronic display device, said wiring extending within said right-atrial catheter.

4. The long-term internal temperature measurement device of claim 1 wherein said electronic display device comprises a thermometry module operative to provide a digital readout display of the patient's temperature and a graphical display of the patient's temperature over time.

5. The long-term internal temperature measurement device of claim 4 wherein said thermometry module further comprises programming to enable a care giver to set upper and lower limits for the patient's temperature and, should those limits be exceeded, said thermometry module is further operative to trigger an alarm to inform the care giver of the change in the patient's temperature.

6. The long-term internal temperature measurement device of claim 1 wherein said electronic display device comprises a hand-held electronic thermometer for intermittent temperature measurement.

7. A long-term internal temperature measurement device comprising:
a silastic, tunneled right-atrial catheter including a distal end adapted to be directed into the superior vena cava of a patient to reside within one of the superior vena cava and the right atrium of the patient's heart, an outer end extending outside of the patient and a cuff positionable beneath the skin of the patient for generally preventing bacterial growth up the catheter;
a thermistor device mounted within said catheter, specifically within said distal end of said catheter, said thermistor device operative to measure the temperature of blood flowing through one of the patient's right atrium and superior vena cava; and
a thermometry module in information transmission connection with said thermistor device, said thermometry module operative to provide a digital readout display of the patient's temperature and a graphical display of the patient's temperature over time as measured by said thermistor device within one of the patient's right atrium and superior vena cava.

8. The long-term internal temperature measurement device of claim 7 wherein said thermistor device comprises a resistor operative to measure temperature changes within the body of the patient by measuring the change in resistance as the temperature varies to determine the specific temperature of the surrounding body fluid.

9. The long-term internal temperature measurement device of claim 7 further comprising electrically conductive wiring extending between and connecting said thermistor device and said thermometry module for transfer of temperature information from said thermistor device to said thermometry module, said wiring extending within said right-atrial catheter.

10. The long-term internal temperature measurement device of claim 7 wherein said thermometry module further comprises programming to enable a care giver to set upper and lower limits for the patient's temperature and, should those limits be exceeded, said thermometry module is further operative to trigger an alarm to inform the care giver of the change in the patient's temperature.
